(19) United States
(12) Patent Application Publication
(10) Pub. No.: US 2024/0241071 A1
(43) Pub. Date: Jul. 18, 2024

(54) GAS HEAT CONDUCTION TYPE HYDROGEN SENSOR HAVING INTEGRATED STRUCTURE

(71) Applicant: WITHMEMS CO., LTD., Hwaseong-si Gyeonggi-do (KR)

(72) Inventors: Jong-Uk BU, Seongnam-si Gyeonggi-do (KR); In-Hoe KIM, Yongin-si Gyeonggi-do (KR); Hwang-Sub KOO, Suwon-si,Gyeonggi-do (KR); Ja-Bung GU, Yongin-si Gyeonggi-do (KR)

(73) Assignee: WITHMEMS CO., LTD., Hwaseong-si Gyeonggi-do (KR)

(21) Appl. No.: 18/562,070

(22) PCT Filed: Jun. 14, 2022
(86) PCT No.: PCT/KR2022/008419

§ 371 Date: Nov. 17, 2023

(30) Foreign Application Priority Data
Jun. 24, 2021 (KR) 10-2021-0082120

(51) Int. Cl. G01N 27/18; G01N 33/00

(52) U.S. Cl. CPC G01N 27/18; G01N 33/005

(57) ABSTRACT

Disclosed is a hydrogen sensor having an integrated structure, the sensor being usable even in poor conditions, having a greatly reduced volume, minimizing the influence of humidity, and facilitating mass production thereof at low cost.

### TECHNICAL FIELD

The present disclosure relates to a gas heat conduction type hydrogen sensor having an integrated structure.

### BACKGROUND ART

With the rapid progress of technology development for energy production using hydrogen ultimately as new clean energy, the recent production output of a fuel cell electric vehicle (FCEV) is rapidly increasing.

A fuel cell electric vehicle (FCEV) is a 100% pollution-free vehicle that is in operation by electricity generated through bonding between hydrogen stored in the vehicle and air in the atmosphere. The fuel cell electric vehicle is provided with a device for converting chemical energy into electrical energy instead of a fuel tank of an existing vehicle.

The fuel cell electric vehicle is provided with a fuel cell stack, a hydrogen supply device, an air supply device, a heat management device, and hydrogen storage device.

Among them, the hydrogen supply and storage devices correspond to a system for storing hydrogen corresponding to the fuel of the fuel cell electric vehicle and transporting a fixed amount of fuel to a stack. In order to manage the hydrogen being supplied, they require monitoring and management of a hydrogen pressure, an overall temperature change, hydrogen leakage, and the like.

A hydrogen sensor is divided into a hydrogen gas leak detection sensor for detecting the leak of the hydrogen gas and a hydrogen concentration sensor for managing the hydrogen concentration. The hydrogen gas leak detection sensor is being applied to the vicinity of a hydrogen storage container, the vicinity of a seam of a hydrogen transport piping system, stack surroundings, and a vehicle interior, and the hydrogen concentration sensor is applied to the vicinity of a stack outlet or the vicinity of a hydrogen dilution and exhaust system.

In particular, the hydrogen gas leak detection sensor is a technology to directly detect the hydrogen gas, and is absolutely necessary to prepare for an explosion risk by the hydrogen compressed with high pressure in the hydrogen tank.

The hydrogen gas detection technology is briefly classified into a hot-wire semiconductor type, a contact combustion type, and a gas heat conduction type, and as types being in the research and development stage, there are an optical type, a field effect transistor (FET) type, a composite material penetration thin film type, and the like.

The hot-wire semiconductor type is to measure the change of an electrical resistance due to gas adsorption on a metal oxide semiconductor surface as the change of a resistance value that appears at both ends of a metal wiring. Further, the contact combustion type is composed of two elements of a detection specimen that reacts on a flammable gas and a compensation specimen that does not react on the flammable gas, and measures a temperature increase of the detection specimen by means of a resistance difference from the compensation specimen when being exposed to the flammable gas. Further, the gas heat conduction type is to measure the temperature change of a heating element due to the heat conduction of the gas.

The above-described types differ from one another depending on the hydrogen concentration, and as illustrated in FIG. 1, the hot-wire semiconductor type is used to detect low-concentration hydrogen, and the contact combustion type is used to detect high-concentration hydrogen.

The contact combustion type hydrogen sensor has an advantage to being able to detect the high-concentration hydrogen, but has a problem with a long-term reliability due to deterioration of a catalyst.

As an alternative to this, a gas heat conduction type hydrogen sensor that can be used to measure the high-concentration hydrogen has been proposed. Since the conductivity of a gas or vapor is a physical property, the quality deterioration or toxification of the catalyst does not occur, and a stable state can be maintained for a long time.

A commercially available hydrogen sensor is provided with a compensation specimen and a detection specimen, constitutes a membrane having heat isolation through silicone micromachining, and is mounted on each of a package having an open cap and a package having a closed cap. Since the hydrogen sensor having the above-described constitution uses two different individual packages together, the volume of the sensor can't help but be large.

In particular, the hydrogen gas leak detection sensor is being applied to the vicinity of a hydrogen storage container, the vicinity of a seam of a hydrogen transport piping system, stack surroundings, and a vehicle interior, and there are limits in mounting the hydrogen sensor in a limited space in the actual vehicle. Further, there are limits in lowering the price of two individually packaged sensors.

Meanwhile, depending on the external environment, and particularly, in case of high humidity, the heat conductivity of the hydrogen sensor is changed due to the water vapor, and thus measurement errors occur in the hydrogen sensor. Accordingly, a method for separately performing humidity correction through attachment of an additional humidity sensor in addition to the hydrogen sensor has been proposed, but this brings a new problem of increased costs.
(Patent Document 1) KR Patent Application Publication No. 10-2015-0030495 (Published on Mar. 20, 2015)
(Patent Document 2) KR Patent Application Publication No. 10-2017-0114985 (Published on Oct. 16, 2017)

### SUMMARY OF INVENTION

### Technical Problem

The inventors have produced a sensor using the principle that heat conductivity of a hydrogen gas is relatively greater than that of other gases in a manner that not only a sensing element and a reference element are formed into one chip and installed in the same package to reduce the sensor volume and to simplify the production process as well but also a heater capable of generating heat is installed inside the chip so that sensing is possible at a temperature that is not affected by the humidity to heighten the response characteristic and accuracy of the hydrogen sensor.

Accordingly, an object of the present disclosure is to provide a gas heat conduction type hydrogen sensor having an integrated structure.

### Solution to Problem

In order to detect hydrogen in a gas heat conduction type, the present disclosure provides a hydrogen sensor having an integrated structure that includes a housing in which a stem and a cap are bonded to accommodate a chip therein.

The chip includes: a substrate; two membranes formed on the substrate to be spaced apart from each other at a predetermined interval and configured to form a sensing element and a reference element, respectively; a heater formed in a center area of the respective membranes and configured to generate Joule heat through heating up to a sensing temperature; an electrode pad formed to be spaced apart from the membranes and the heater for a predetermined distance; and at least one open hole formed in a predetermined area of the stem corresponding to the sensing element so that the sensing element comes in contact with a gas.

A diameter D of the open hole H satisfies Equation 1 below.

$$D < ( a + 2 T ) / ( tan θ ) \qquad [ Equation 1 ]$$

(In the above equation,

D represents the diameter of the open hole, a represents a length of a membrane side of the sensing element,

T represents a thickness of the substrate, and

θ is an angle equal to or less than 90 degrees.)

The substrate has a structure in which a rear surface thereof is etched so that the sensing element and the reference element have a heat isolation structure.

The membrane is a single-layer or multilayer thin film including at least one of silicon oxide (SiOₓ), silicon nitride (SiNₓ), and silicon oxynitride (SiOₓN_{y}).

The heater is able to heat up to a temperature equal to or greater than 400° C.

One or more of an air and an inert gas are injected into an inner area composed of the chip and the cap.

Further, the present disclosure provides a method for manufacturing a hydrogen sensor having an integrated structure, which includes:
(S1) forming a membrane by depositing and then etching an insulation film on a substrate;
(S2) forming a heater by forming and then etching a conductive thin film on the membrane;
(S3) forming an electrode pad by depositing and then etching an electrode material on the substrate;
(S4) etching a rear surface of the substrate on which the membrane is not formed so that a sensing element and a reference element have a heat isolation structure thereon;
(S5) preparing a stem having more than one open hole H in a predetermined area;
(S6) mounting a chip on the stem; and
(S7) boding the stem and a cap together.

### Advantageous Effects of Invention

The gas heat conduction type hydrogen sensor according to the present disclosure can detect the hydrogen gas.

Since the hydrogen sensor has the integrated structure provided with the sensing element and the detection element in one package, it has a drastically reduced volume in comparison to the existing sensor including two individual packages, and thus it can be very easily mounted in the limited indoor space.

Further, the hydrogen sensor can be easily manufactured with the production cost greatly reduced, and thus it is competitive in comparison to similar products.

In addition, since the effects of humidity can be excluded by increasing selectivity to the humidity through raising the temperature of the heater provided in the sensing element and the reference element over a specified temperature, the hydrogen sensor can be used even without a separate sensor for humidity compensation.

### BRIEF DESCRIPTION OF DRAWINGS

FIG. 1 illustrates a hydrogen sensor that can be used depending on hydrogen concentration.

FIG. 2 is a cross-sectional view of a hydrogen sensor according to the present disclosure.

FIG. 3A is a front view of a chip according to the present disclosure, and FIG. 3B is a cross-sectional view taken along line Q-Q′ of a chip according to the present disclosure.

FIG. 4 is a graph illustrating heat conductivity of a fluid depending on a temperature.

FIG. 5A is a front view of a chip according to an implementation example of the present disclosure, and FIG. 5B is a photograph of a chip according to an implementation example of the present disclosure.

FIG. 6 is a diagram of a circuit configured to identify the characteristic of a hydrogen sensor of the present disclosure.

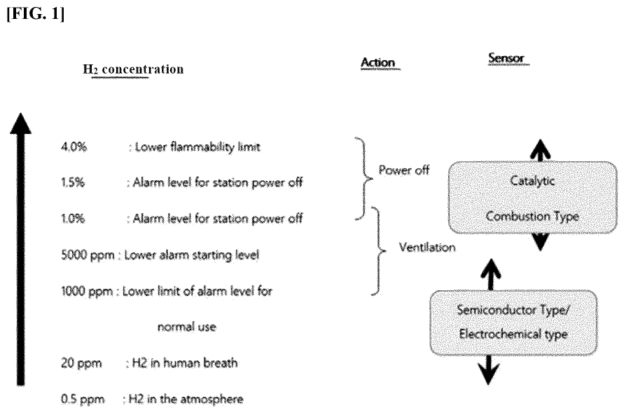

### BEST MODE OF INVENTION

In order to detect hydrogen in a gas heat conduction type, according to one implementation example of the present disclosure, a hydrogen sensor having an integrated structure may include a housing in which a stem and a cap are bonded to accommodate a chip therein, wherein the chip includes: a substrate; two membranes formed on the substrate to be spaced apart from each other at a predetermined interval and configured to form a sensing element and a reference element, respectively; a heater formed in a center area of the respective membranes and configured to generate Joule heat through heating up to a sensing temperature; an electrode pad formed to be spaced apart from the membranes and the heater for a predetermined distance; and at least one open hole formed in a predetermined area of the stem corresponding to the sensing element so that the sensing element comes in contact with a gas.

Here, a diameter D of the open hole H may satisfy Equation 1 below:

$$D < ( a + 2 T ) / ( tan θ ) \qquad [ Equation 1 ]$$

(in the above equation,

D represents the diameter of the open hole,

a represents a length of a membrane side of the sensing element,

T represents a thickness of the substrate, and

θ represents an angle equal to or less than 90 degrees.)

### DESCRIPTION OF EMBODIMENTS

A heat conduction type hydrogen sensor according to the present disclosure has been designed to reduce the volume of the sensor and not be influenced by external environmental factors other than hydrogen, and particularly, by humidity, at the same time.

The sensor volume can be reduced by forming a sensing element and a reference element into one chip and installing the same in the same package, and the factors caused by the external environment, that is, the humidity, can be solved by installing a heater element in the chip.

Hereinafter, the hydrogen sensor according to the present disclosure will be described in more detail with reference to the drawings.

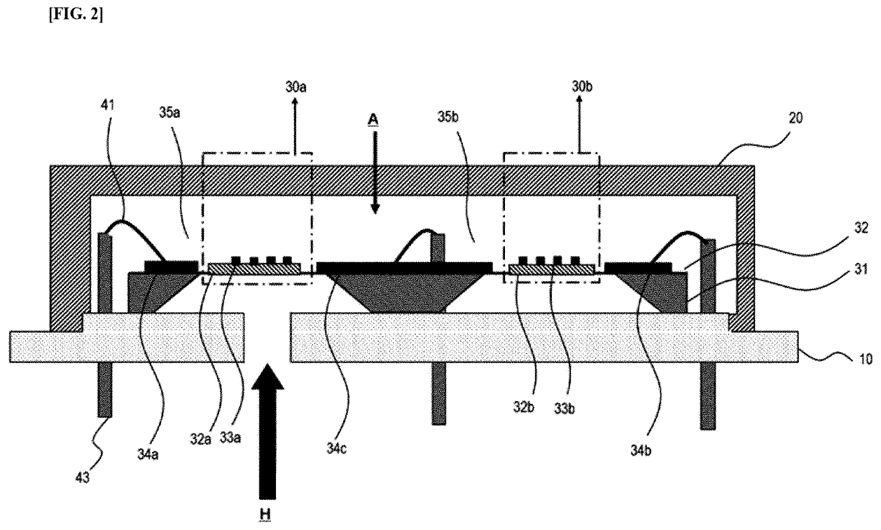

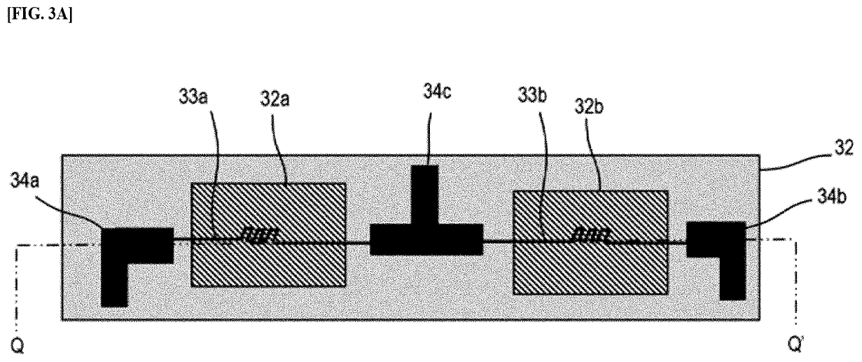

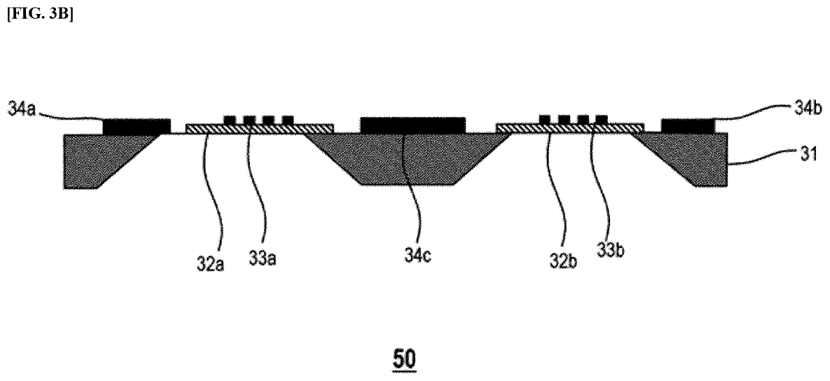

FIG. 2 is a cross-sectional view of a hydrogen sensor according to the present disclosure. FIG. 3A is a front view of a chip according to the present disclosure, and FIG. 3B is a cross-sectional view taken along line Q-Q′ of a chip according to the present disclosure.

Referring to FIGS. 2 and 3, the hydrogen sensor is composed of a housing through bonding of a stem 10 and a cap 20 in order to accommodate a chip 50 therein.

The hydrogen sensor has a structure in which a package is formed by die bonding on the stem 10, and the cap 20 is bonded inside the sensor in order to prevent external gases from flowing therein.

The chip 50 is formed in a center part of the stem 10, and a plurality of through-holes are provided therein so that a plurality of connector pins 43 can pass through the through-holes, respectively.

The cap 20 is formed to cover the chip 50 mounted on the stem 10, and although the shape of the cap 20 is not limited, it has a cylindrical shape, and is fastened with the stem 10.

The chip 50 includes membranes 32a and 32b, heaters 33a and 33b, and electrode pads 34a, 34b, and 34c, which respectively form a sensing element 30a and a reference element 30b on a substrate.

A silicon substrate may be used as a substrate 31, and if necessary, a glass, sapphire, or quartz substrate may be used. In this case, a rear surface of a center area of the substrate 31 on which the heaters 33a and 33b are formed may have a structure having been removed through etching, that is, a heat isolation structure in which the sensing element and the reference element are heat-isolated.

A pair of membranes 32a and 32b are formed so that the sensing element 30a and the reference element 30b are able to be formed, and are positioned to be spaced apart from each other at a predetermined interval and to face each other.

Although the sizes of the membrane 32a that forms the sensing element 30a and the membrane 32b that forms the reference element 30b may be equal to or different from each other, it is preferable that the sizes are equal to each other.

The membranes 32a and 32b can be made of a material having heat resistance as well as mechanical properties, serve as anti-etching layers when the rear surface of the substrate 31 is etched, and serve as supports of the heaters 33a and 33b. Further, the membranes 32a and 32b can prevent the chip from being deformed by heating during heating of the heaters 33a and 33b. Preferably, the membranes 32a and 32b are laminated to include at least one of silicon oxide (SiOₓ), silicon nitride (SiNₓ), and silicon oxynitride (SiOₓN_{y}). As an example, the membranes 32a and 32b may be in the form of a multilayer thin film, such as silicon oxide/silicon nitride/silicon oxide.

Meanwhile, the hydrogen sensor according to the present disclosure is provided with the heaters 33a and 33b in the chip 50 in order to exclude the factors caused by the external environment, that is, the humidity. More specifically, the heater provided in the sensing element and the reference element can exclude the influence exerted by the humidity by increasing selectivity to the humidity through raising the temperature of the heater provided in the sensing element and the reference element over a specified temperature.

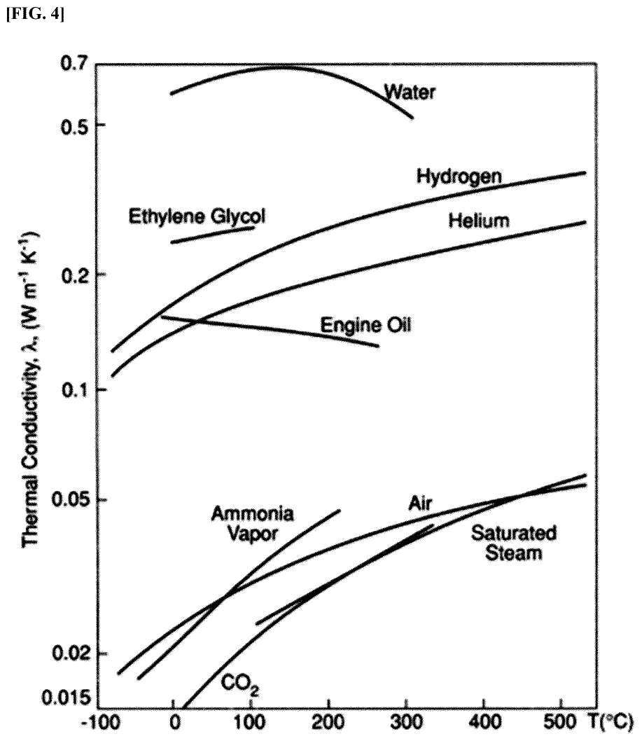

FIG. 4 is a graph illustrating heat conductivity of a fluid depending on a temperature, and shows various specific tendencies that the heat conductivities of various fluids increase or decrease depending on the temperature. Among them, water is represented as a curve on which the heat conductivity increases up to a temperature of about 150° C., and then decreases thereafter, and it can be known that the water is rapidly vaporized over about 350° C., and thus does not exert an influence exerted on the heat conductivity.

The gas heat conduction type hydrogen sensor detects the hydrogen gas through a difference in heat conductivity, and in case of heating over the temperatures of FIG. 4, the influence of the humidity against the hydrogen sensor can be completely excluded.

Accordingly, in the present disclosure, as illustrated in FIGS. 2 and 3, it can be known that the heat conductivity change of the hydrogen sensor by the humidity can be minimized by mounting the heaters 33a and 33b that can heat the water over the vaporization temperature thereof in the chip 50 and generating Joule heat through the operation of the heaters 33a and 33b.

The heaters 33a and 33b are disposed in center areas of the respective membranes 32a and 32b so that the Joule heat generation is performed with respect to all of the sensing element 30a and the reference element 30b. The Joule heat may be generated by applying a voltage to both ends of the heaters 33a and 33b, and the Joule heat is generated up to at least 250° C. that is higher than the evaporation temperature of the water, and preferably, up to 400° C. or more. As a result, the sensing can be stably performed at this temperature, and thus this temperature may be the sensing temperature of the hydrogen sensor.

A material that can be used as the heaters 33a and 33b may be a metal or a semiconductor oxide, and preferably, may be a metal material, and more preferably, may be any one of gold, tungsten, platinum, and palladium.

The overall length, thickness, and shape of the heaters 33a and 33b are adjusted to have a designed resistance, and specifically, a resistance in the range of 500 to 1000Ω, and preferably, the heaters 33a and 33b are formed in an inter-digital shape or a gap shape.

If necessary, in order to increase adhesion during forming of the heaters 33a and 33b, an adhesive layer (not illustrated) using chrome (Cr) or titanium (Ti) may be further formed on the membranes 32a and 32b. The adhesive layer may be formed by using a method, such as a sputtering method, an electron beam method, or a vaporization method.

In addition, in order to minimize the influence by humidity, a humidity sensor may be further provided, and may perform humidity compensation of the hydrogen sensor through the measured humidity.

The electrode pads 34a, 34b, and 34c are formed to be spaced apart from the membranes 32a and 32b and the heaters 33a and 33b for predetermined distances, and are manufactured by using a material having the characteristic that is the same as or similar to the characteristic of the heaters 33a and 33b. The electrode pads 34a, 34b, and 34c may serve to transfer a power to the heaters 33a and 33b, and a bonding wire 41 for connection with a power supply source may come in contact with the electrode pads.

The bonding wire 41 may be a conductive wire, and may make the electrode pads 34a, 34b, and 34c and a printed circuit board be electrically connected to each other. Accordingly, the heater resistance signal sensed by the sensing element 30a is transferred to the printed circuit board through the electrode pads 34a, 34b, and 34c and the bonding wire 41. As the bonding wire 41, a known wire, such as a gold wire, an aluminum wire, or a copper wire, may be used.

In particular, according to the hydrogen sensor of the present disclosure, at least one open hole H for making a gas flow into the sensing element 30a is formed. In this case, although one open hole H is illustrated in FIG. 2 for convenience, two or more open holes can be installed.

The open hole H is formed in an area of the stem 10 corresponding to the sensing element 30a to facilitate an inflow of a hydrogen gas that is an identification target in the gas, but the hydrogen gas is made not to flow into the reference element 30b. Accordingly, the gas having passed through the open hole H of the stem 10 flows to the inside of the hydrogen sensor, but the inflow of the gas into the reference element 30b is blocked by the substrate 31 patterned in the form of a partition wall through etching of a lower area of the substrate 31 at a predetermined angle as in FIG. 2.

The open hole H has a vertical partition of a predetermined level so as to facilitate an inflow/outflow to the sensing element 30a, and although a single open hole H or a plurality of open holes H are installed, the overall forming area of the open hole H is made not to exceed the width of the membrane 32a positioned on the sensing element 30a.

More preferably, the diameter D of the open hole H satisfies Equation 1 below.

$$D < ( a + 2 T ) / ( tan θ ) \qquad [ Equation 1 ]$$

(in the above equation,

D represents the diameter of the open hole,

a represents a length of a membrane side of the sensing element,

T represents a thickness of the substrate, and

θ represents an angle equal to or less than 90 degrees.)

The substrate 31 has a shape in which the cross-sectional area of the upper part is large, and the cross-sectional area becomes smaller as going toward the lower part, and in this case, an angle θ formed by the stem 10 and the substrate 31 can be controlled through an etching process of the substrate 31. Preferably, θ is equal to or below 90 degrees, and preferably, is an angle of 54.74 degrees or an angle in the range of 85 to 90 degrees, and specifically, is 54.74 degrees.

Further, the side length of the membrane 32a, which is defined as a, may be a length of a small side of the membrane 32a in a horizontal direction.

In case of designing the diameter of the open hole H to satisfy Equation 1, the sensitivity of the hydrogen gas can be increased.

The open hole H may have a horizontal cross section that is in the form of a circle, a rectangle, or a polygon, and the shape of the open hole H is not specially limited in the present disclosure.

Meanwhile, one or more of an air and an inert gas may be injected into an inner area A composed of the chip 50 and the cap 20, and an external gas is made not to flow into the inner space A. Preferably, noise caused by other gases can be minimized through filling with the inert gas.

A plurality of connector pins 43 may be formed and bonded onto a printed circuit board (not illustrated) through soldering, and thus may transfer an electrical signal through the printed circuit board to an external electronic device. The connector pin 43 may be made of nickel, copper, or an alloy thereof.

If necessary, an insulation film (not illustrated) may be formed in the form of covering specified areas of the electrode pads 34a, 34b, and 34c, the heaters 33a and 33b, or the membranes 32a and 32b, but forming of the insulation film is not essential.

According to the hydrogen sensor according to the present disclosure as constituted above, mass production of sensors is facilitated through sensor integration.

Specifically, a method for manufacturing a chip in a hydrogen sensor according to the present disclosure includes:
(S1) forming membranes 32a and 32b by depositing and then etching an insulation film on a substrate 31;
(S2) forming heaters 33a and 33b by forming and then etching a conductive thin film on the membranes 32a and 32b;
(S3) forming electrode pads 34a, 34b, and 34c by depositing and then etching an electrode material on the substrate 31; and
(S4) etching a rear surface of the substrate 31 on which the membranes 32a and 32b are not formed so that a sensing element and a reference element have a heat isolation structure thereon.

First, the membranes 32a and 32b are formed by depositing and then etching the insulation film on the substrate 31 (S1).

The insulation film is a material for forming the membranes 32a and 32b, and is laminated as a single layer or a multilayer and includes at least one of silicon oxide (SiOₓ), silicon nitride (SiNₓ), and silicon oxynitride (SiOₓN_{y}). As the lamination method, a dry method may be used to form the insulation film by using a method, such as thermal oxidation, sputtering, or chemical vapor deposition.

Next, the heaters 33a and 33b are formed by forming and then etching a conductive thin film on the membranes 32a and 32b (S2).

The conductive thin film may be made of a metal or a semiconductor oxide, and preferably, may be made of one or more of gold, tungsten, platinum, and palladium. The conductive thin film can be formed by using a method, such as a sputtering method, an electron beam method, or an evaporation method. The etching may be performed in a photolithography process that is used in the semiconductor process.

Next, the electrode pads 34a, 34b, and 34c are formed by depositing and then etching the electrode material on the substrate 31 (S3).

Any material having conductivity can be used as the electrode material, and is manufactured by using a material having the characteristic that is the same as or similar to the characteristic of the heaters 33a and 33b. As an example, the electrode material may be one or more of gold, tungsten, platinum, and palladium. The electrode material can be deposited in the method, such as the sputtering method, the electron beam method, or the evaporation method. The etching may be performed in the photolithography process that is used in the semiconductor process.

Next, the rear surface of the substrate 31 on which the membranes 32a and 32b are not formed is etched so that the sensing element and the reference element have the heat isolation structure (S4).

The etching may be performed through a dry etching process using a photoresist pattern. As an example, opening patterning for silicon etching is performed by using a double side exposure, and a wet anisotropic etching using a solution, such as KOH, TMAH, and EDP, or a dry etching using a silicon deep RIE device may be performed.

If the substrate 31 is produced in the form of an island having the heat isolation structure by partially removing the lower part of the substrate 31, the sensitivity to the gases flowing into the hydrogen sensor can be further increased.

The hydrogen sensor is produced in a manner that the chip 50 manufactured through the above-described steps passes through the steps of:
(S5) preparing the stem 10 provided with more than one open hole H in a predetermined area;
(S6) mounting the chip 50 on the stem 10; and
(S7) boding the stem 10 and the cap 20 together.

In the step S5, the forming of the open hole H of the stem 10 is not specially limited in the present disclosure, and various known perforation method may be used.

However, the open hole H has a vertical partition of a predetermined level so as to facilitate an inflow/outflow to the sensing element 30a, and although a single open hole H or a plurality of open holes H are installed, the overall forming area of the open hole H is made not to exceed the width of the membrane 32a positioned on the sensing element 30a.

More preferably, the diameter D of the open hole H satisfies Equation 1 below.

$$D < ( a + 2 T ) / ( tan θ ) \qquad [ Equation 1 ]$$

(in the above equation,

D represents the diameter of the open hole,

a represents a length of a membrane side of the sensing element,

T represents a thickness of the substrate, and

θ represents an angle equal to or less than 90 degrees.)

The substrate 31 has a shape in which the cross-sectional area of the upper part is large, and the cross-sectional area becomes smaller as going toward the lower part, and in this case, the angle θ formed by the stem 10 and the substrate 31 can be controlled through an etching process of the substrate 31. Preferably, 0 is equal to or below 90 degrees, and preferably, is an angle of 54.74 degrees or an angle in the range of 85 to 90 degrees, and specifically, is 54.74 degrees.

Further, the side length of the membrane 32a, which is defined as a, may be a length of a small side of the membrane 32a in a horizontal direction.

In case of designing the diameter of the open hole H to satisfy the Equation 1, the sensitivity of the hydrogen gas can be increased.

The open hole H may have a horizontal cross section that is in the form of a circle, a rectangle, or a polygon, and the shape of the open hole H is not specially limited in the present disclosure.

Next, the chip 50 is mounted on the stem 10, and along with this, it is connected to the bonding wire 41 through soldering, so as to perform an electrical connection to an outside through the connector pins 43.

The bonding in S7 is not specially limited in the present disclosure, and a known method may be used. However, if necessary, a step of injecting an air or an inert gas into the inner area formed by the chip 50 and the cap 20 may be further performed.

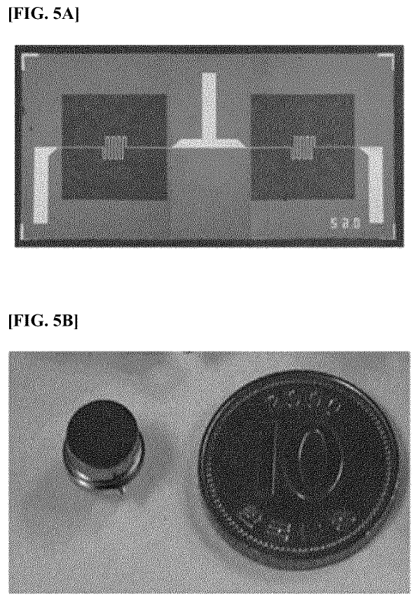

FIG. 5A is a front view of a chip 50 according to an implementation example of the present disclosure, and FIG. 5B is a photograph thereof.

As shown in FIG. 5A, the chip 50 of the hydrogen sensor according to the present disclosure is provided with the sensing element 30a and the reference element 30b, and the hydrogen sensor can be produced with a volume that is smaller than a coin as shown in FIG. 5B.

The hydrogen sensor according to the present disclosure can detect a hydrogen gas by a gas heat conduction method.

The detection of the hydrogen gas is performed after the temperature of the hydrogen sensor is increased by the heaters 33a and 33b. If the gas including the hydrogen gas comes in contact with the sensing element 30a, the temperature of the sensing element 30a gets down due to a difference of heat conductivity of hydrogen. Accordingly, the resistance of the heater 33a formed in the area of the sensing element 30a is changed, and thus not only detection of the hydrogen gas but also measurement of the concentration of the hydrogen gas can be performed through measurement of the resistance change against the resistance of the heater 33b formed in the area of the reference element 30b.

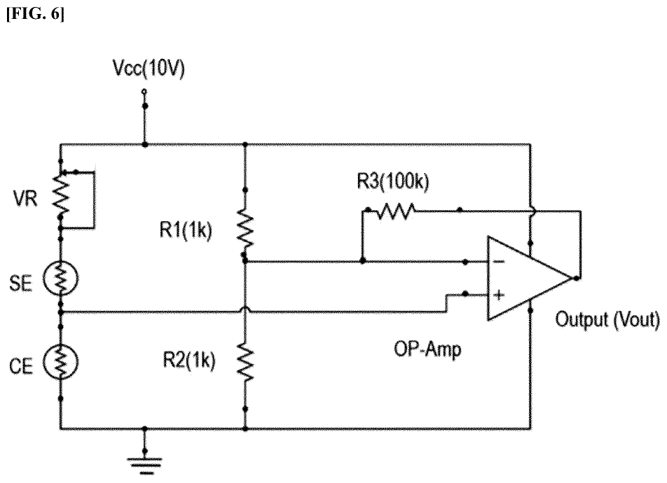

FIG. 6 is a diagram of a circuit configured to identify the characteristic of a hydrogen sensor of the present disclosure.

Referring to FIG. 6, the hydrogen sensor may be briefly composed of a bridge circuit including four resistors of fixed resistors R1, R2, and R3 and a variable resistor VR, and a power V being applied to the bridge circuit. In this case, if the resistance of the heaters 33a and 33b is set to 800Ω, the resistance change by the hydrogen gas is detected as an electrical signal through the bridge circuit including the sensing element 30a and the reference element 30b.

The hydrogen concentration can be estimated through the principle that as the hydrogen flows into the sensing part 30a, the heat conductivity of the hydrogen differs, and thus the temperature of the sensing element 30a gets down to cause the resistance of the heater to be changed.

As a result, the hydrogen sensor according to the present disclosure showed a fast response speed, and it appeared that a recovery time of about tens of seconds was necessary to get back again when the hydrogen concentration was lowered after the hydrogen detection. The characteristic of such a response speed has an excellent numerical value that is equal to or better than that of other expensive sensors.

Further, in order to detect the hydrogen gas leakage, the hydrogen sensor of the present disclosure can be applied to the vicinity of a hydrogen storage container, the vicinity of a seam of a hydrogen transport piping system, stack surroundings, and a vehicle interior in the fuel cell electric vehicle.

In particular, the hydrogen sensor according to the present disclosure has the integrated structure provided with the sensing element and the detection element in one package, thereby drastically reducing volume in comparison to the existing sensor including two individual packages, and thus it can be very easily mounted in the limited indoor space.

Further, the hydrogen sensor can be easily manufactured with the production cost greatly reduced, and thus it is competitive in comparison to similar products.

In addition, since the effects of humidity for the hydrogen sensor can be excluded by the heaters provided therein, the hydrogen sensor can be used even without a separate sensor for humidity correction, and if necessary, it can be installed around the installation position of the hydrogen sensor.

Although the present disclosure has been described with reference to the limited embodiment and drawings, it will be apparent to those of ordinary sill in the art to which the present disclosure pertains that various modifications are possible within the range of the technical ideas of the present disclosure. Accordingly, the scope of the present disclosure should be determined by the description of the claims and their equivalents.

[Description of Symbols]

| | |
|---|---|
| 10: stem | 20: cap |
| 30a: sensing element | 30b: reference element |
| 32a, 32b: membrane | 33a, 33b: heater |
| 34a, 34b, 34c: electrode pad | |
| 41: bonding wire | |
| 43: connector pin | 50: chip |

### INDUSTRIAL APPLICABILITY

The present disclosure relates to a gas heat conduction type hydrogen sensor having an integrated structure, which is applicable to a fuel cell electric vehicle and the like.

1. A hydrogen sensor having an integrated structure comprising:
a housing in which a stem and a cap are bonded to accommodate a chip therein,
wherein the chip includes:
a substrate;
two membranes formed on the substrate to be spaced apart from each other at a predetermined interval and configured to form a sensing element and a reference element, respectively;
a heater formed in a center area of the respective membranes and configured to generate Joule heat through heating up to a sensing temperature;
an electrode pad formed to be spaced apart from the membranes and the heater for a predetermined distance; and
at least one open hole formed in a predetermined area of the stem corresponding to the sensing element so that the sensing element comes in contact with a gas.

2. The hydrogen sensor of claim 1, wherein a diameter D of the open hole H satisfies Equation 1 below:
$$D < ( a + 2 T ) / ( tan θ ) \qquad [ Equation 1 ]$$
(In the above equation,
D represents the diameter of the open hole,
a represents a length of a membrane side of the sensing element,
T represents a thickness of the substrate, and
θ is an angle equal to or less than 90 degrees.)

3. The hydrogen sensor of claim 1, wherein the substrate has a structure in which a rear surface thereof is etched so that the sensing element and the reference element have a heat isolation structure.

4. The hydrogen sensor of claim 1, wherein the membrane is a single-layer or multilayer thin film including at least one of silicon oxide (SiOₓ), silicon nitride (SiNₓ), and silicon oxynitride (SiOₓN_{y}).

5. The hydrogen sensor of claim 1, wherein the heater is able to heat up to a temperature equal to or greater than 400° C.

6. The hydrogen sensor of claim 1, wherein one or more of an air and an inert gas are injected into an inner area composed of the chip and the cap.

7. A method for manufacturing a hydrogen sensor having an integrated structure, the method comprising:
(S1) forming a membrane by depositing and then etching an insulation film on a substrate;
(S2) forming a heater by forming and then etching a conductive thin film on the membrane;
(S3) forming an electrode pad by depositing and then etching an electrode material on the substrate;
(S4) etching a rear surface of the substrate on which the membrane is not formed so that a sensing element and a reference element have a heat isolation structure thereon;
(S5) preparing a stem having more than one open hole H in a predetermined area;
(S6) mounting a chip on the stem; and
(S7) boding the stem and a cap together.

8. The method of claim 7, wherein a diameter D of the open hole H satisfies Equation 1 below:
$$D < ( a + 2 T ) / ( tan θ ) \qquad [ Equation 1 ]$$
(In the above equation,
D represents the diameter of the open hole,
a represents a length of a membrane side of the sensing element,
T represents a thickness of the substrate, and
θ is an angle equal to or less than 90 degrees.)
